(19) United States
(12) Patent Application Publication
(10) Pub. No.: US 2024/0382548 A1
(43) Pub. Date: Nov. 21, 2024

(54) COMPOSITIONS COMPRISING CASTANEA SATIVA EXTRACTS FOR TREATMENT OF HELICOBACTER PYLORI INFECTIONS

(71) Applicant: ISTITUTO FARMOCHIMICO FITOTERAPICO EPO S.R.L. ED IN FORMA ABBREVIATA: EPO S.R.L., Milano (IT)

(72) Inventors: Giovanna Nicotra, Milano (IT); Silvia Vicentini, Milano (IT); Maria Grazia Marano, Milano (IT); Beatrice Bruno, Milano (IT); Violetta Insolia, Milano (IT); Mario Dell'Agli, Milano (IT); Enrico Sangiovanni, Milano (IT); Giulia Martinelli, Milano (IT); Stefano Piazza, Milano (IT)

(73) Assignee: ISTITUTO FARMOCHIMICO FITOTERAPICO EPO S.R.L. ED IN FORMA ABBREVIATA: EPO S.R.L., Milano (IT)

(21) Appl. No.: 18/689,907

(22) PCT Filed: Sep. 6, 2022
(86) PCT No.: PCT/IB2022/058345

§ 371 Date: Mar. 7, 2024

(30) Foreign Application Priority Data
Sep. 9, 2021 (IT) 102021000023282

(51) Int. Cl. A61K 36/49; A61P 31/04

(52) U.S. Cl. CPC A61K 36/49; A61P 31/04; A61K 2236/31

(57) ABSTRACT

Extracts of the leaves, bark, and seeds/fruits of Castanea sativa Mill., syn. Castanea vesca Gaertn., and extracts of aerial parts of Cistus x incanus L., possess antibacterial activity against Helicobacter pylori and anti-inflammatory activity at the gastric level, and are therefore useful to relieve gastrointestinal inflammations, in particular those caused by Helicobacter pylori.

The object of the present invention is the use of Castanea sativa extracts, optionally combined with Cistus x incanus extracts, for the treatment of Helicobacter pylori infections.

### PRIOR ART

Gastritis is an inflammatory disorder mainly caused by the infection of the bacterium Helicobacter pylori, which colonises the gastric mucosa of about 80% of the population in developing countries and about 50% of the global population [Rugge et al., Digestive and liver disease: official journal of the Italian Society of Gastroenterology and the Italian Association for the Study of the Liver 2011. 43 Suppl 4]. Helicobacter pylori only colonises human gastric mucosa; in vitro reproduction of the human epithelial infection process, therefore, constitutes a useful research tool. The infection can induce an immune response in the host, characterised by the release of numerous inflammatory cytokines into the gastric mucosa. The exposure of the gastric epithelial cells to cytokines, mainly TNFα and IL-1β, as well as the pathogen, induces the release of IL-8, a potent chemokine that promotes neutrophils' infiltration. IL-8 secretion is a distinctive feature of gastritis induced by Helicobacter pylori [Shimada and Terano, J Gastroenterol 1998. 33(5)], and its release is closely associated with the activation of NF-κB, a transcription factor involved in various pathophysiological processes, including inflammatory processes, cell growth, and proliferation. The infusion obtained from chestnut leaves is a traditional remedy against cough, while the decoction of leaves or roots was used for diarrhoea [Chevallier A., The Encyclopedia of Medicinal Plants, Dorling Kindersley, 1996]. Cistus is traditionally used for respiratory and gastric disorders, as an antibacterial and antioxidant, but there are no publications regarding its specific antibacterial activity against Helicobacter pylori. Plants rich in tannins have been traditionally used to treat gastric ulcers; tannins have also exhibited antibacterial activities in vitro against Helicobacter pylori [Funatogawa et al., Microbiology and immunology 2004.48] and inhibition of gastric release of IL-8, both in vitro and in vivo [Fumagalli et al. Pharmacological research: 2016;111; Sangiovanni et al., PloS one 2013;8(8)].

Castanea sativa Mill., syn. Castanea vesca Gaertn., fam. Fagaceae, is a rich source of tannins, especially in the leaves, wood, and bark. In particular, chestnut leaves contain ellagitannins, a particular class of hydrolysable tannins, to which castalagin and vescalagin belong. One recent study shows an anti-inflammatory effect of chestnut in vitro, but in the context of LPS-induced neuronal inflammation [Chiocchio et al., Metabolites 2020; 10(10)]. However, no studies appear to have investigated the antibacterial effect on H. pylori, whereas studies of other Gram-positive and Gram-negative bacteria have been published [Basile et al., Fitoterapia. 2000; 71 Suppl 1:S110-6. Quave et al., PLoS One 2015;10(8). Silva et al., Antioxidants (Basel) 2020;9(1)].

Cistus x incanus L., fam. Cistaceae, is an evergreen shrub typical of maquis shrubland. Infusions obtained from various species belonging to the genus Cistus have traditionally been used to relieve disorders of the digestive and respiratory tracts, and for some skin problems. Its antioxidant properties [Gori et al., 2016 Int J Mol Sci. 2016 Aug. 17;17(8):1344] and its activity against Gram-positive bacteria [Kuchta et al., Cardiol J. 2019 Mar. 26] are also well known, but its specific antibacterial activity against Helicobacter pylori is unknown; only one published study demonstrates the anti-ulcer activity of an aqueous extract (i.e. different from the hydroalcoholic extract) obtained by decoction of the aerial parts, in rats with ulcers induced by HCl/EtOH, indomethacin, or reserpine [Attaguile et al., Pharmacol Res. 1995;31(1):29-32]. Studies of its anti-inflammatory activity are also very limited [Mansoor et al., Nat. Prod. Res. 2016; 30(17): 1919-26].

### DESCRIPTION OF THE INVENTION

It has now been found that extracts of Castanea sativa Mill., syn. Castanea vescaGaertn., and extracts of aerial parts of Cistus x incanus L., have antibacterial activity against Helicobacter pylori and anti-inflammatory activity at the gastric level and are therefore useful to relieve gastrointestinal inflammations, in particular those caused by Helicobacter pylori.

Castanea sativa (chestnut) extracts and Cistus x incanus (cistus) extracts are traditionally used for completely different purposes: the former as an antioxidant and to promote the fluidity of the bronchial secretions as well as to regularise intestinal transit (due to its astringent properties); and the latter, not only as an antioxidant, but also to generally promote the digestive and respiratory functions (Annex 1 to the Ministerial Decree of 10 Aug. 2018 governing the use of plant substances and preparations in food supplements, as amended by the Decree of 9 Jan. 2019).

The first object of the invention, therefore, relates to extracts of leaves, fruits, or bark of Castanea sativa for use in the treatment of Helicobacter pylori infections.

The second object of the invention relates to compositions comprising an extract of leaves, fruits, or bark of Castanea sativa and an extract of aerial parts of Cistus x incanus.

The extracts for the uses and compositions according to the invention are preferably aqueous or hydroethanolic extracts.

The chestnut extract is preferably titrated in ellagitannins, in particular, castalagin and vescalagin; an extract with a weight content of castalagin and vescalagin ranging between 1 and 6% is particularly preferred.

This extract can be obtained through a process that includes the following steps: the parts of the plant, in particular the leaves, are pre-dried and chopped, preferably to a length of 1-2 cm and they are treated with an extractive solvent containing water, ethanol, or mixtures thereof; this mixture is kept under percolation; at the end of the first recycling, the extraction process is repeated from 3 to 5 times under the same conditions, using a fresh solvent every time. All the extracts are combined, filtered, and concentrated; the solvent is removed by a rotary evaporator under vacuum and heating, and then the extract is dried to obtain a homogeneous brown powder.

The castalagin and vescalagin content is determined by HPLC-DAD on a C18 (250×4.6 mm) 5 μm Purospher endcapped column. A solution of a 0.100 g sample of dried extract in DMSO is diluted with methanol, and the resulting sample is injected, after filtration through an 0.45 μm nylon filter, in the amount of 10 μl at a flow rate of 0.8 ml/min, using a gradient of water with 0.1% formic acid:methanol as mobile phase. The revelation is taken at the wavelength of 254 nm in comparison with standard solutions of 1 mg/ml of castalagin and vescalagin dissolved in DMSO.

The cistus extract has a polyphenol content ranging between 18 and 25% preferably, and it can be obtained through a process that includes the following steps: the aerial parts, are pre-dried and chopped, preferably to a length of 1-2 cm, and they are treated with a solvent containing water, ethanol, or mixtures thereof; this mixture is kept under percolation and under heating; at the end of the first recycling, the extraction process is repeated from 3 to 5 times under the same conditions, using a fresh solvent every time. The extracts are combined, filtered, and concentrated; the solvent is removed by a rotary evaporator under vacuum and heating, and then the extract is dried to obtain a homogeneous brown powder.

The compositions according to the invention can be formulated with suitable vehicles and excipients in various forms, such as food supplements, functional foods, and beverages.

The compositions according to the invention comprise from 20 mg to 100 mg per dosage unit of Castanea sativa extract and from 20 mg to 100 mg of Cistus x incanus extract.

The anti-inflammatory and antibacterial effects of the chestnut extract, the ingredients thereof and the compositions according to the invention are described in the following experimental part.

### Anti-Inflammatory and Antibacterial Effect on Helicobacter pylori of Chestnut Extract, Vescalagin, and Castalagin

Firstly, the extract was analysed to establish its possible cytotoxicity (using the MTT colorimetric assay) towards non-tumoral human gastric epithelial cells GES-1 in the presence of H. pylori (ratio 1:50). The extract did not exhibit cytotoxicity after 6 hours' treatment at the tested concentrations (10, 25, 50, and 100 μg/ml).

At the same concentrations, the extract inhibits IL-8 release induced by H. pylori in GES-1 cells; the pathogen induces IL-8 release in gastric epithelial cells, of about a 20-fold increase compared with the unstimulated control. The chestnut extract proved to inhibit chemokine release after 6 hours' treatment. The IC50 of the extract ranged between 20.38 μg/ml and 41.19 μg/ml. EGCG 50 μM (50% inhibition) was used as a positive control.

Castalagin and vescalagin did not exhibit any toxicity in the 10-50 μM concentration range. The same concentrations were used to evaluate the effect on IL-8, exhibiting concentration-dependent inhibition of the chemokine release induced by the pathogen, with comparable IC50 values.

### Effect of the Extracts on Nuclear Transcription Factor NF-κB

The nuclear transcription factor NF-κB is involved in inflammation of the gastric epithelium induced by H. pylori and is also responsible for increased expression of pro-inflammatory genes, including IL-8, in the gastric cells. An evaluation was therefore conducted to establish whether the extract inhibits the said transcription factor, thereby demonstrating one of the possible molecular mechanisms underlying the inhibitory effect on the release of IL-8, whose expression is dependent on NF-κB. The cells were 20 subjected to transient transfection with a plasmid containing three KB responsive sites upstream of the gene encoding for luciferase; the cells then underwent treatment with the pathogen, and the transcription was measured after 6 hours by luminescence.

The pathogen induces an approximately two-fold increase in NF-κB-guided transcription; the chestnut extract, whether digested or undigested, exhibits inhibition of transcription (100 μg/ml), and consequently anti-inflammatory activity. EGCG (epigallocatechin gallate) 50 μM (63% inhibition) was used as a positive control.

An evaluation of the pathogen-induced NF-κB-guided transcription demonstrates that, in samples subjected to gastric digestion, the 50:50 mixture of the two extracts (100 μg/ml) exhibits an inhibitory activity of 26%, whereas the extracts, at the concentration at which they are present in the mixture (50 μg/ml), are either inactive (cistus extract) or exhibit very low activity (9%), as in the case of the chestnut extract. This finding further demonstrates the synergic effect of the two ingredients.

The immunofluorescence analysis of NF-κB translocation demonstrates that chestnut significantly reduces the said factor induced by H. pylori, at the concentrations of both 50 and 100 μg/ml, whereas the cistus extract does not exhibit any activity against the said parameter. The quantification of the immunofluorescence intensity of NF-κB at nuclear level demonstrates that chestnut (100 μg/ml) restores the translocation levels to values comparable with those of cells not inflamed by H. pylori (−80%). The combination of cistus and chestnut extracts retains inhibitory activity towards NF-κB translocation.

### Antibacterial Activity of Chestnut Extract, Castalagin, and Vescalagin on H. pylori

The antibacterial activity of the chestnut extract was evaluated by measuring the MIC (Minimum Inhibitory Concentration) on a strain of H. pylori. The extract shows a MIC of about 100 μg/ml, and therefore exhibits statistically significant inhibition of the growth of the pathogen starting from this concentration. Tetracycline (0.25 μg/mL), which inhibits 80% of the growth of the pathogen, was used as a positive control.

Under the same experimental conditions, castalagin and vescalagin exhibit an inhibitory effect on the growth of the pathogen, with a comparable MIC corresponding to 25 μM.

### Anti-Inflammatory Effect (IL-8 Release) and Antibacterial Effect on Helicobacter pylori of a Cistus Extract and of the Combined Chestnut Extract and Cistus Extract

The extract of C. incanus, subjected to simulated gastric digestion and the cell viability assay (MMT assay in GES-1 cells), did not exhibit cytotoxicity either before or after gastric digestion at the tested concentrations. The extract exhibited an inhibitory action on IL-8 release to a concentration-dependent extent, both before and after digestion; moreover, when subjected to simulated gastric digestion, the extract maintained an inhibitory activity comparable with that of the undigested extract (IC₅₀ of undigested extract 23.99 μg/ml, and of digested extract 31.95 μg/ml).

The C. incanus extract exhibits a MIC of 125 μg/ml (comparable to that of chestnut) against H. pylori before digestion, and 250 μg/ml after simulated gastric digestion.

The combination in equal parts (50:50) of chestnut leaf extract and C. incanus extract is devoid of cytotoxicity, and exhibits a particularly interesting inhibitory effect on IL-8 (IC₅₀ 24.41 (μg/ml) and MIC 125 μg/ml. The results are reported in Table 1 below.

Moreover, the evaluation of the ability of the extracts to inhibit adherence of the bacteria to human gastric epithelium, the first mechanism whereby the pathogen induces the cascade of toxic events at the gastric level, demonstrates a particularly interesting effect of the cistus extract at a concentration of only 25 μg/ml, whereas the chestnut leaf extract does not exhibit any significant effect at the same concentration, or even at the concentration of 50 μg/ml. A mixture consisting of cistus and chestnut extracts (50:50), assayed at the concentration of 100 μg/ml on antiadhesive activity, exhibits an inhibitory effect of 65%, whereas the individual cistus and chestnut extracts exhibit 40% and 15% inhibition respectively, confirming the superiority of the combination over the individual extracts, when considered separately.

In conclusion, the cistus extract exhibits an interesting antiadhesive activity, not attributable to the chestnut extract. Conversely, chestnut exhibits more marked anti-inflammatory activity, involving the transcription factor NF-κB. The combination of the two extracts exhibits synergic activity in the treatment of gastric inflammations related to H. pylori infection.

TABLE 1
Anti-inflammatory and antibacterial activity of undigested extracts
of chestnut, cistus, and the combination thereof (C + C).

| ANTI-INFLAMMATORY | IC₅₀ | IC₅₀ | IC₅₀ |
|---|---|---|---|
| ACTIVITY | CHESTNUT | CISTUS | C + C |
| (IL-8) | 20.38-41.19 μg/ml | 23.99 μg/ml | 24.41 μg/ml |

| ANTIBACTERIAL | MIC | MIC | MIC |
|---|---|---|---|
| ACTIVITY | CHESTNUT | CISTUS | C + C |
| H. pylori | 100 μg/ml | 125 μg/ml | 125 μg/ml |

1. A method of treatment Helicobacter pylori infections in patients in need thereof, said method comprising:
administering to said patients a pharmaceutical effective amount of an extract of Castanea sativa.

2. The method according to claim 1, wherein the extract is titrated in ellagitannins.

3. The method according to claim 2, wherein the extract has a castalagin and vescalagin content by weight ranging between 1 and 6%.

4. The method according to claim 1, wherein the extract is obtained from leaves, bark, seeds/fruits.

5. Compositions comprising fruit, leaves, or bark extract of Castanea sativa and extract of aerial parts of Cistus x incanus L.

6. Compositions according to claim 5, wherein the Castanea sativa extract has a castalagin and vescalagin content by weight ranging between 1 and 6%.

7. Compositions according to claim 5, wherein the extract of aerial parts of Cistus x incanus L. has a polyphenol content ranging between 18 and 25%.

8. Compositions according to claim 5, in the form of supplements, functional foods, and beverages.
